(19) United States
(12) Patent Application Publication
(10) Pub. No.: US 2024/0212802 A1
(43) Pub. Date: Jun. 27, 2024

(54) MAPPING ELECTRONIC MESSAGES TO LABORATORY RESULT IDENTIFIERS TO INFORM PATIENT TREATMENT AND CLINICAL TRIAL DESIGN

(71) Applicant: Flatiron Health, Inc., New York, NY (US)

(72) Inventors: Kim Peifer, Philadelphia, PA (US); Ryan Steier, Houston, TX (US); Anna Bolha, New York, NY (US); Chase Wang, New York, NY (US); Xin Tan, New York, NY (US); Ivan Quan, New York, NY (US)

(21) Appl. No.: 18/391,460

(22) Filed: Dec. 20, 2023

Related U.S. Application Data
(60) Provisional application No. 63/435,522, filed on Dec. 27, 2022.

(51) Int. Cl. G16H 10/40; G16H 50/20

(52) U.S. Cl. CPC G16H 10/40; G16H 50/20

(57) ABSTRACT

Described herein are methods and systems for receiving electronic messages comprising laboratory results and associating the laboratory results with appropriate laboratory result identifiers to generate mapped laboratory results. The mapped laboratory results may be used to inform clinical patient treatment and/or clinical trial design or performance.

### RELATED APPLICATION DATA

This application claims the benefit of priority of U.S. Provisional Patent Application Ser. No. 63/435,522, filed Dec. 27, 2022, and titled “Mapping Electronic Messages To Laboratory Result Identifiers To Inform Patient Treatment And Clinical Trial Design,” which is incorporated by reference herein in its entirety.

### BACKGROUND

Clinical laboratory results are widely used in healthcare systems to treat patients and design and conduct clinical trials. However, conventional methods of identifying laboratory results in electronic messages can be prone to errors. Accordingly, improved methods and systems for mapping laboratory results in electronic messages to laboratory result identifiers are needed.

### SUMMARY

Methods and systems for mapping laboratory results in electronic messages to laboratory result identifiers are generally described.

In some aspects, a method comprises using at least one hardware processor to perform: receiving an electronic message comprising a laboratory result segment comprising a laboratory result; identifying a standard laboratory result identifier associated with the laboratory result segment; and associating the laboratory result segment with the standard laboratory result identifier to generate a mapped laboratory result segment.

In some aspects, a method comprises using at least one hardware processor to perform: receiving a plurality of electronic messages, wherein a first electronic message of the plurality of electronic messages comprises a laboratory result segment comprising a laboratory result; generating a request for a map between one or more field values of the laboratory result segment and a standard laboratory result identifier; receiving, in response to the request, the map between the one or more field values and the standard laboratory result identifier; associating the laboratory result segment with the standard laboratory result identifier to generate a first mapped laboratory result segment; and associating one or more additional laboratory result segments that comprise the one or more field values with the standard laboratory result identifier to generate one or more additional mapped laboratory result segments.

In some aspects, a system comprises a message ingestion module configured to receive an electronic message. In some embodiments, the system comprises a message parsing module configured to receive the electronic message from the message ingestion module and parse the electronic message into one or more field values. In certain embodiments, at least one of the one or more field values comprises a laboratory result. In some embodiments, the system comprises an identifier extraction module configured to receive the one or more field values from the message parsing module and identify a standard laboratory result identifier associated with the laboratory result.

In some aspects, a system comprises a message ingestion module configured to receive an electronic message. In some embodiments, the system comprises a message parsing module configured to receive the electronic message from the message ingestion module and parse the electronic message into one or more field values. In certain embodiments, at least one of the one or more field values comprises a laboratory result. In some embodiments, the system comprises an identifier extraction module configured to receive the one or more field values from the message parsing module and identify any laboratory result identifiers associated with the laboratory result. In some embodiments, the system comprises a mapping request module configured to receive the one or more field values and generate a request to map at least one of the one or more field values to a standard laboratory result identifier.

### BRIEF DESCRIPTION OF DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1 is a flow diagram of an exemplary method for mapping laboratory results in electronic messages to standard laboratory result identifiers, according to some embodiments.

FIG. 2 is a block diagram of an exemplary system for mapping laboratory results in electronic messages to standard laboratory result identifiers, according to some embodiments.

FIG. 3 is a flow diagram of an exemplary method for mapping laboratory results in electronic messages that do not comprise laboratory result identifiers to standard laboratory result identifiers, according to some embodiments.

FIG. 4 is a block diagram of an exemplary system for mapping laboratory results in electronic messages that do not comprise laboratory result identifiers to standard laboratory result identifiers, according to some embodiments.

### DETAILED DESCRIPTION

Described herein are methods and systems for receiving electronic messages comprising laboratory results and associating the laboratory results with appropriate laboratory result identifiers to generate mapped laboratory results. The mapped laboratory results may be used to inform clinical patient treatment and/or clinical trial design or evaluation.

Clinical laboratory results may be useful for a wide range of applications, including clinical patient treatment, clinical trials, and clinical or other scientific research (e.g., research using well coded patient data sets). For example, a healthcare provider may rely at least in part on one or more clinical laboratory results to diagnose a patient with a disease, select an appropriate treatment (e.g., prescribe a pharmaceutical drug), modify an aspect of a selected treatment (e.g., adjust a dosage of a prescribed pharmaceutical drug), and/or evaluate patient outcomes. As another example, researchers designing and/or conducting a clinical trial to test the toxicity and/or efficacy of a medical treatment (e.g., a pharmaceutical drug, a medical device) may rely at least in part on one or more clinical laboratory results to define a cohort, evaluate performance and/or tolerability of the medical treatment, and/or generate real world evidence for the clinical trial.

A system (e.g., an electronic health record (EHR) system, a clinical trial design assessment system) may receive electronic messages comprising laboratory results from a variety of sources. For example, the electronic messages may be sent by one or more laboratory test vendors (e.g., Quest Diagnostics, Labcorp, Orchard Laboratories, BioReference Laboratories) and/or one or more healthcare facilities capable of conducting and analyzing clinical laboratory tests (e.g., hospitals, healthcare clinics, doctors' offices). An electronic message comprising a laboratory result may include a standard laboratory result identifier, a source-specific laboratory result identifier (e.g., a vendor-specific laboratory result identifier), or no identifier at all.

Given the different potential sources of electronic messages and the lack of consistency in methods of coding laboratory results in electronic messages, it is possible for some laboratory results in electronic messages to be incorrectly mapped (e.g., by identifying a laboratory result in an electronic message as an incorrect type of laboratory result, failing to identify an electronic message as comprising a laboratory result, etc.). Incorrect mapping can have serious negative consequences, including patients receiving a suboptimal dose of a medication and/or being inappropriately included in or excluded from a clinical trial. Even seemingly minor errors in mapping can have significant impacts. As an illustrative example, incorrect mapping of a creatinine value from a urine sample of a cancer patient as a creatinine value from a blood sample of the cancer patient may result in the cancer patient being prescribed an incorrect dosage of a chemotherapy medication due to an incorrect dosage calculation. In some cases, incorrect mapping may result in incorrect information being transmitted to patients, healthcare providers, cancer registries, and/or immunization registries. In some cases, incorrect mapping may result in incorrect conclusions being drawn regarding a medical treatment's toxicity and/or efficacy in real world and clinical trial settings.

Methods and systems described herein advantageously provide accurate mapping of laboratory results in electronic messages to laboratory result identifiers. Such accurate mapping may allow healthcare providers to have a complete and longitudinal view of laboratory results, regardless of where laboratory tests were performed, and may inform appropriate clinical treatment of patients and accurate patient outcome analysis.

In some aspects, a method comprises receiving an electronic message comprising a laboratory result segment comprising a laboratory result. In some embodiments, the method comprises identifying a standard laboratory result identifier associated with the laboratory result. In some embodiments, the method further comprises associating the laboratory result with the standard laboratory result identifier to generate a mapped laboratory result. In some embodiments, one or more steps of the method may be performed using at least one hardware processor.

In some aspects, a method comprises receiving an electronic message comprising a laboratory result segment comprising a laboratory result. In some embodiments, the method comprises identifying a standard laboratory result identifier associated with the laboratory result segment. In some embodiments, the method further comprises associating the laboratory result segment with the standard laboratory result identifier to generate a mapped laboratory result segment. In some embodiments, one or more steps of the method may be performed using at least one hardware processor.

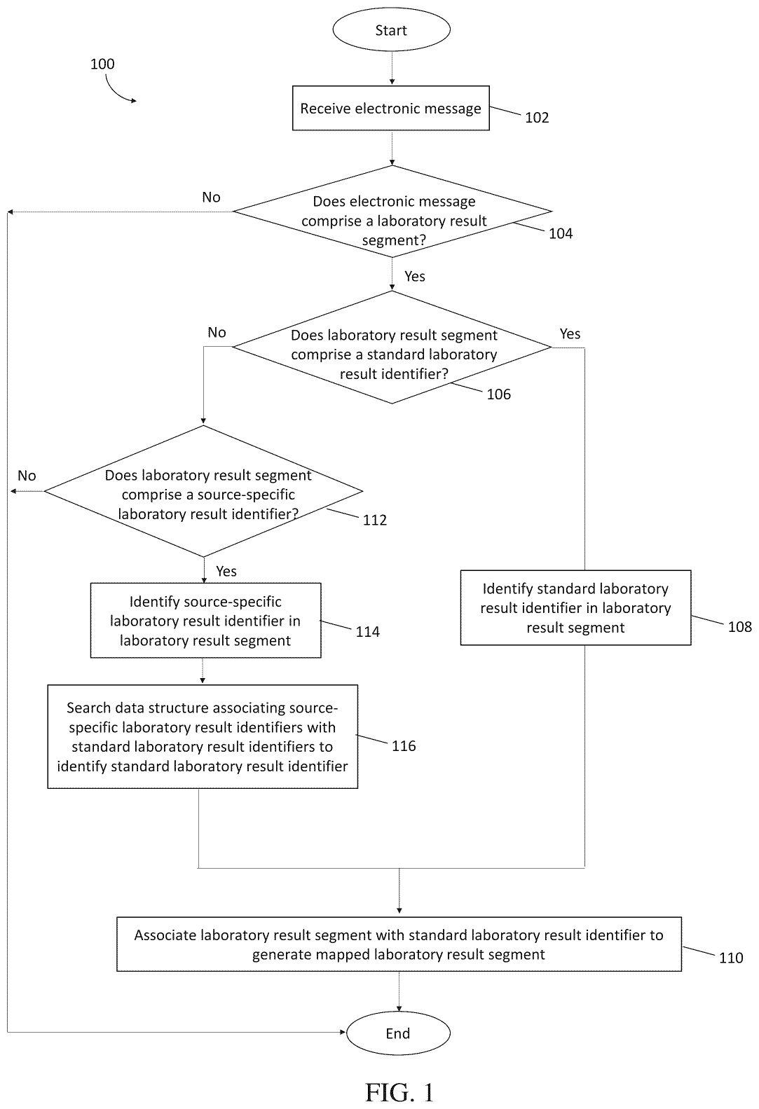

FIG. 1 shows a flow diagram of exemplary method 100 of mapping a laboratory result in an electronic message to a standard laboratory result identifier, according to some embodiments. In some embodiments, some or all steps of method 100 may be performed using at least one hardware processor.

Method 100 begins at block 102, which comprises receiving the electronic message. In some embodiments, the electronic message is received by a system (e.g., an electronic health record (EHR) system, a clinical trial design assessment system) from a laboratory test vendor (e.g., Quest Diagnostics, Labcorp, Orchard Laboratories, BioReference Laboratories) or a healthcare facility (e.g., hospital, healthcare clinic, doctor's office). The electronic message may conform to one or more messaging standards. Examples of messaging standards include, but are not limited to, Health Level 7 (HL7), Fast Healthcare Interoperability Resources (FHIR), Continuity of Care Record (CCR), Continuity of Care Document (CCD), and Cross Enterprise Document Sharing (XDS) standards. In certain embodiments, the electronic message is an HL7 message. In some instances, the HL7 message is an HL7 v2.x message. Non-limiting examples of HL7 v.2x messages include HL7 v.2.2, 2.3, 2.3.1, 2.4, 2.5, 2.5.1, 2.6, 2.7, 2.7.1, 2.8, 2.8.1, 2.8.2, and 2.9 messages. In some instances, the electronic message is an HL7 v3.x message. In certain cases, the electronic message is encrypted or otherwise secured.

In some embodiments, the electronic message (e.g., an HL7 message) comprises one or more segments. Non-limiting examples of segments include Observation Result (OBX), Observation Request (OBR), Specimen Information (SPM), Message Header (MSH), Patient Identification (PID), Common Order Segment (ORC), Event Type (EVN), Software (SFT), and Notes and Comments (NTE) segments. In certain embodiments, the one or more segments comprise an Observation Result (OBX) segment. In some embodiments, the OBX segment comprises a laboratory result. In certain embodiments, at least one (and, in some cases, each) segment comprises one or more fields. In some instances, two or more fields within a segment may be separated by vertical pipe symbols. In certain embodiments, at least one field of at least one segment comprises one or more sub-fields. In some instances, two or more sub-fields within a field may be separated by caret symbols. In some embodiments, at least one (and, in some cases, each) of the one or more fields comprises a field value (e.g., information associated with the field, including information associated with any sub-field, sub-sub-field, etc. of the field).

In certain embodiments, at least one field value comprises a laboratory result. The laboratory result may be the result of any clinical laboratory test (e.g., a test to determine the presence, absence, or quantity of an analyte in any type of specimen from a human or animal subject). In certain embodiments, at least one field value comprises a laboratory result identifier. In some embodiments, the laboratory result identifier is a standard laboratory result identifier. As used herein, a standard laboratory result identifier refers to an identifier encoded according to a laboratory test identification standard. A non-limiting example of a laboratory test identification standard is Logical Observation Identifier Names and Code (LOINC). In certain embodiments, the standard laboratory result identifier is a LOINC code. In some cases, a LOINC code may be associated with one or more attributes. Examples of attributes that may be associated with a LOINC code include a “component” or “analyte” (e.g., the substance or entity being measured or observed), “system” or “specimen” (e.g., the specimen or thing upon which the observation was made), “property” (e.g., the characteristic or attribute of the analyte), “time” (e.g., the interval of time over which an observation was made), “scale” (e.g., how the observation value is quantified or expressed, such as “quantitative,” “ordinal,” or “nominal”), and “method” (e.g., how the observation was made). In some embodiments, the laboratory result identifier is a source-specific laboratory result identifier. As used herein, a source-specific laboratory result identifier refers to an identifier associated with a source of the laboratory result (e.g., a laboratory vendor).

As an illustrative example, an Observation Result (OBX) segment of an HL7 message may comprise a plurality of fields, including but not limited to a third field (OBX-3) comprising an observation identifier (e.g., a laboratory result identifier) and a fifth field (OBX-5) comprising an observation value (e.g., a laboratory result). In some cases, the third field OBX-3 comprises a first sub-field (OBX-3.1) comprising an identifier associated with a laboratory test, a second sub-field (OBX-3.2) comprising the name of the laboratory test, and a third sub-field (OBX-3.3) comprising the name of the coding system of the identifier (e.g., “LN,” “LOINC,” and/or “LNC” for a LOINC code, “L” for a local, source-specific identifier). In some cases, the third field OBX-3 comprises a fourth sub-field (OBX-3.4) comprising an alternate identifier associated with the laboratory test, a fifth sub-field (OBX-3.5) comprising alternate text, and a sixth sub-field (OBX-3.6) comprising the name of the coding system of the alternate identifier.

After receiving the electronic message, method 100 proceeds to block 104, which comprises determining whether the electronic message comprises a laboratory result segment. A laboratory result segment generally refers to a portion of an electronic message comprising a laboratory result. For example, in an HL7 message, a laboratory result segment may comprise an OBX segment (and, in some cases, one or more additional segments, such as an associated OBR and/or NTE segment). Accordingly, in certain embodiments where the electronic message is an HL7 message, determining whether the electronic message comprises a laboratory result segment comprises determining whether the electronic message comprises one or more OBX segments. If one or more OBX segments are present, it may be determined that the electronic message comprises a laboratory result segment. If no OBX segments are present, it may be determined that the electronic message does not comprise a laboratory result segment. In certain cases, one or more non-OBX segments of an electronic message may be used in addition or as an alternative to OBX segments to determine whether the electronic message comprises a laboratory result segment.

In some cases, an electronic message may comprise two or more laboratory result segments (e.g., OBX segments). In some such cases, each laboratory result segment (e.g., OBX segment) may separately proceed to the next step of method 100 such that each laboratory result segment is separately mapped to a standard laboratory result identifier.

When it is determined that an electronic message comprises a laboratory result segment, method 100 proceeds to block 106, which comprises determining whether the laboratory result segment comprises a standard laboratory result identifier. In some embodiments, determining whether the laboratory result segment comprises a standard laboratory result identifier comprises searching one or more field values of the laboratory result segment for an occurrence of “LN”, “LOINC”, and/or “LNC.” In certain embodiments where the laboratory result segment comprises an OBX segment, determining whether the laboratory result segment comprises a standard laboratory result identifier comprises identifying a third field OBX-3 of the OBX segment and searching a third sub-field OBX-3.3 and/or a sixth sub-field OBX-3.6 of the third field OBX-3 for an occurrence of “LN,” “LOINC,” and/or “LNC.” In certain embodiments, the third sub-field OBX-3.3 may initially be searched for an occurrence of “LN,” “LOINC,” and/or “LNC,” and if no such occurrence is found, the sixth sub-field OBX-3.6 may be searched. In some cases, one or more fields and/or sub-fields of a laboratory result segment other than OBX-3.3 and OBX-3.6 may be searched for an occurrence of “LN,” “LOINC,” and/or “LNC.” In certain instances, each field of a laboratory result segment (e.g., an OBX segment) may be searched for an occurrence of “LN,” “LOINC,” and/or “LNC.” If an occurrence of “LN,” LOINC,” or “LNC” is found in the laboratory result segment, it may be determined that the laboratory result segment comprises a standard laboratory result identifier. If no occurrence of “LN,” “LOINC,” or “LNC” is found in the laboratory result segment, it may be determined that the laboratory result segment does not comprise a standard laboratory result identifier.

When it is determined that the laboratory result segment comprises a standard laboratory result identifier, method 100 proceeds to block 108, which comprises identifying the standard laboratory result identifier (e.g., LOINC code) in the laboratory result segment. In certain embodiments where the laboratory result segment comprises an OBX segment and the third sub-field OBX-3.3 was found to comprise an occurrence of “LN,” “LOINC,” and/or “LNC,” the field value contained in a first sub-field OBX-3.1 of the third field OBX-3 may be identified as the standard laboratory result identifier. In certain embodiments where the laboratory result segment comprises an OBX segment and the sixth sub-field OBX-3.6 was found to comprise an occurrence of “LN,” “LOINC,” and/or “LNC,” the field value contained in a fourth sub-field OBX-3.4 of the third field OBX-3 may be identified as the standard laboratory result identifier. In some instances, identifying the standard laboratory result identifier comprises searching one or more other fields (or subfields thereof) of the laboratory result segment (e.g., fields/sub-fields other than OBX-3.1 or OBX-3.4). In some instances, the standard laboratory result identifier may be recognized based on a standard format of the standard laboratory result identifier.

When it is determined that the laboratory result segment does not comprise a standard laboratory result identifier (e.g., no field value comprises an occurrence of “LN,” “LOINC,” and/or “LNC”), method 100 proceeds to block 112, which comprises determining whether the laboratory result segment comprises a source-specific laboratory result identifier. In certain embodiments where the laboratory result segment comprises an OBX segment, determining whether the laboratory result segment comprises a source-specific laboratory result identifier comprises searching a third sub-field OBX-3.3 and/or a sixth sub-field OBX-3.6 of the third field of the OBX segment for an occurrence of the character “L” (e.g., “L\hat{ }”, “L|”). In some instances, the third sub-field OBX-3.3 may initially be searched for an occurrence of “L” (e.g., “L\hat{ }”, “L|”), and if no such occurrence is found, the sixth sub-field OBX-3.6 may be searched. In some embodiments, determining whether the laboratory result segment comprises a source-specific laboratory result identifier comprises searching one or more other fields (or subfields thereof) of the laboratory result segment (e.g., fields/sub-fields other than OBX-3.3 or OBX-3.6). In certain instances, each field of a laboratory result segment (e.g., an OBX segment) may be searched for an occurrence of “L” (e.g., “L\hat{ }”, “L|”). In some embodiments, determining whether the laboratory result segment comprises a source-specific laboratory result identifier comprises searching one or more fields (or subfields thereof) of the laboratory result segment for a different arrangement of characters (e.g., other than “L\hat{ }” or “L|”) indicating the presence of a source-specific laboratory result identifier in the laboratory result segment.

When it is determined that the laboratory result segment comprises a source-specific laboratory result identifier, method 100 proceeds to block 114, which comprises identifying the source-specific laboratory result identifier in the laboratory result segment. In some embodiments where an occurrence of “L” (e.g., “L\hat{ }”, “L|”) was found in the third sub-field OBX-3.3, identifying the source-specific laboratory result identifier comprises identifying the field value contained in a first sub-field OBX-3.1 of the third field OBX-3 of the OBX segment as the source-specific laboratory result identifier. In some embodiments where an occurrence of “L” (e.g., “L\hat{ }”, “L|”) was found in the sixth sub-field OBX-3.6, identifying the source-specific laboratory result identifier comprises identifying the field value contained in a fourth sub-field OBX-3.4 of the third field OBX-3 of the OBX segment as the source-specific laboratory result identifier. In some instances, identifying the source-specific laboratory result identifier comprises searching one or more other fields (or subfields thereof) of the laboratory result segment (e.g., fields/sub-fields other than OBX-3.1 or OBX-3.4). In some instances, the source-specific laboratory result identifier may be recognized based on a standard format of the source-specific laboratory result identifier.

When the source-specific laboratory result identifier has been identified, method 100 proceeds to block 116, which comprises searching a data structure associating source-specific laboratory result identifiers with standard laboratory result identifiers to identify a standard laboratory result identifier. The data structure may be any data structure suitable for storing and organizing source-specific laboratory result identifiers and standard laboratory result identifiers. Non-limiting examples of suitable data structures include tables, arrays, lists, databases, and the like. In some embodiments, the data structure is stored in the system receiving the electronic messages (e.g., an EHR system, a clinical trial design assessment system, or a subsystem thereof), which may be referred to as a “message-receiving system.” In some embodiments, the data structure is stored in an external system (e.g., a system operated by a laboratory vendor or other third party) in communication with the message-receiving system. In some embodiments, the message-receiving system may communicate with the external system to identify the standard laboratory result identifier using any suitable communication interface and communication protocol. In certain embodiments, the message-receiving system may send a source-specific laboratory result identifier to the external system, and the external system may return the corresponding standard laboratory result identifier.

When the standard laboratory result identifier has been identified, method 100 proceeds to block 110, which comprises associating the laboratory result segment with the standard laboratory result identifier to generate a mapped laboratory result segment. In some embodiments, block 110 further comprises associating the laboratory result segment with a system-specific laboratory result identifier. In some embodiments, the system-specific laboratory result identifier is unique to the message-receiving system (e.g., an EHR system, a clinical trial design assessment system).

In some embodiments, method 100 further comprises using the mapped laboratory result segment during clinical treatment of a patient and/or assessment of one or more aspects of clinical trial design or performance. In some embodiments, the method comprises using the mapped laboratory result segment during clinical treatment of a patient. In certain embodiments, using the mapped laboratory result segment during clinical treatment of a patient comprises diagnosing the patient with a disease, suggesting a treatment for the patient, and/or calculating a dosage of a medication based on the mapped laboratory result segment. In some embodiments, the method comprises using the mapped laboratory result segment during an assessment of one or more aspects of clinical trial design or performance. Clinical trials or studies are generally designed to test the toxicity and/or efficacy of medical treatments (e.g., pharmaceuticals, medical procedures) and/or medical devices. In some embodiments, using the mapped laboratory result segment during an assessment of one or more aspects of clinical trial design or performance comprises determining whether a patient falls within a cohort and/or evaluating one or more effects of a medical treatment on a patient enrolled in a clinical trial based on the mapped laboratory result segment.

In some embodiments, method 100 further comprises identifying one or more laboratory result attributes associated with the standard laboratory result identifier. The one or more laboratory result attributes may comprise component, system, property, time, scale, and/or method. In some embodiments, the standard laboratory result identifier is associated with at least one, at least two, at least three, at least four, at least five, or at least six laboratory result attributes. In some embodiments, the standard laboratory result identifier is associated with one, two, three, four, five, or six laboratory result attributes.

In some embodiments, one or more steps of method 100 may be omitted.

Some aspects are directed to systems for mapping laboratory results in electronic messages to standard laboratory result identifiers. In some embodiments, a system comprises at least one hardware processor and at least one non-transitory computer-readable storage medium storing processor-executable instructions that, when executed by the at least one hardware processor, cause the at least one hardware processor to perform a method described herein (e.g., method 100). In certain embodiments, the system is (or is a component of) an electronic health record (EHR) system and/or a clinical trial design assessment system. In some embodiments, the system comprises a message ingestion module, a message parsing module, and/or an identifier extraction module.

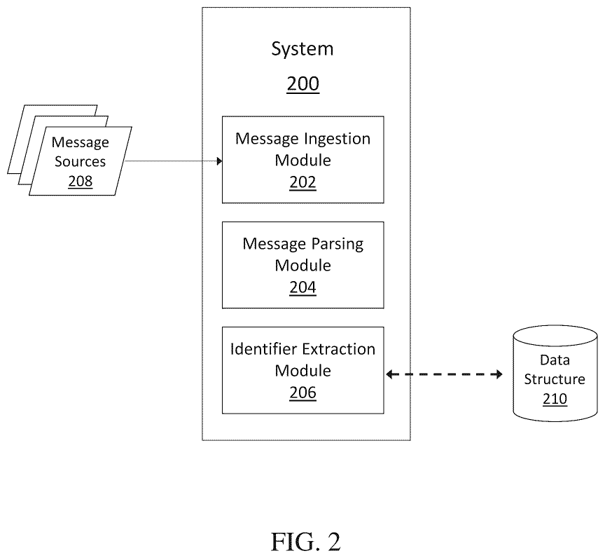

FIG. 2 shows a block diagram of an exemplary system 200 for mapping laboratory results in electronic messages to standard laboratory result identifiers. In some embodiments, system 200 comprises message ingestion module 202, message parsing module 204, and/or identifier extraction module 206.

In some embodiments, message ingestion module 202 is configured to receive electronic messages from message sources 208. Message sources 208 may comprise one or more laboratory vendors and/or one or more healthcare facilities. In some cases, electronic messages may be transmitted from message sources 208 to message ingestion module 202 through a communication network (e.g., the Internet).

In some embodiments, message parsing module 204 is configured to receive electronic messages from message ingestion module 202 and parse the electronic messages into separate components (e.g., segments, field values). In some embodiments, message parsing module 204 is configured to determine whether an electronic message received from message ingestion module 202 comprises one or more laboratory result segments (e.g., a segment or other collection of one or more field values comprising a laboratory result) and to send any laboratory result segments to identifier extraction module 206. In certain embodiments where the electronic message is an HL7 message, message parsing module 204 may be configured to parse the HL7 message into one or more segments and, in some cases, determine whether the one or more segments comprise one or more OBX segments. In instances where the HL7 message comprises one or more OBX segments, each OBX segment (and, in some cases, one or more additional segments, such as associated OBR and/or NTE segments) may be sent to identifier extraction module 206 as a laboratory result segment. In some embodiments, a separate module (not shown in FIG. 2) may be configured to receive the separate components (e.g., segments, field values) of electronic messages from message parsing module 204, determine whether the components comprise a laboratory result segment, and send any laboratory result segments to identifier extraction module 206.

In certain embodiments, identifier extraction module 206 is configured to receive a laboratory result segment from message parsing module 204 (or, in some cases, another module of system 200) and extract any laboratory result identifiers that may be present in the laboratory result segment.

In some instances, identifier extraction module 206 is configured to search the components (e.g., fields, sub-fields, etc.) of the laboratory result segment for an occurrence of “LN,” “LOINC,” and/or “LNC” and identify the associated LOINC code as a standard laboratory result identifier. In certain embodiments where the laboratory result segment comprises an OBX segment, identifier extraction module 206 may be configured to search a third sub-field OBX-3.3 and/or a sixth sub-field OBX-3.6 of a third field OBX-3 for an occurrence of “LN,” “LOINC,” and/or “LNC.” In some embodiments, identifier extraction module 206 may be configured to search one or more other components of the laboratory result segment for an occurrence of “LN,” “LOINC,” and/or “LNC.” If an occurrence of “LN,” “LOINC,” and/or “LNC” is found, identifier extraction module 206 may search one or more components of the laboratory result segment (e.g., first sub-field OBX-3.1 and/or fourth sub-field OBX-3.4 of the third field of the OBX segment) for an associated standard laboratory result identifier (e.g., LOINC code). In some embodiments, identifier extraction module 206 may be configured to search one or more other components of the laboratory result segment for a standard laboratory result identifier. In certain cases, identifier extraction module 206 may be configured to recognize a standard laboratory result identifier based on a standard format of the standard laboratory result identifier.

In some instances, if no occurrence of “LN,” “LOINC,” or “LNC” is found, identifier extraction module 206 may be configured to search the components (e.g., fields, sub-fields, etc.) of the laboratory result segment for a source-specific laboratory result identifier. In certain embodiments where the laboratory result segment comprises an OBX segment, identifier extraction module 206 may be configured to search a third sub-field OBX-3.3 and/or a sixth sub-field OBX-3.6 of a third field OBX-3 for an occurrence of “L” (e.g., “L\hat{ }”, “L|”). In some embodiments, identifier extraction module 206 may be configured to search one or more other components of the laboratory result segment for an occurrence of “L” (e.g., “L\hat{ }”, “L|”). If an occurrence of “L” (e.g., “L\hat{ }”, “L|”) is found, identifier extraction module 206 may search one or more components of the laboratory result segment (e.g., first sub-field OBX-3.1 and/or fourth sub-field OBX-3.4 of the third field of the OBX segment) for an associated source-specific laboratory result identifier. In some embodiments, identifier extraction module 206 may be configured to search one or more (and, in some cases, all) other components of the laboratory result segment for a source-specific laboratory result identifier. In certain cases, identifier extraction module 206 may be configured to recognize a standard laboratory result identifier based on a standard format of the source-specific laboratory result identifier.

In some cases, if identifier extraction module 206 identifies a source-specific laboratory result identifier, module 206 may search data structure 210 associating source-specific laboratory result identifiers with standard laboratory result identifiers (e.g., LOINC codes). In some embodiments, data structure 210 is stored in system 200 or a parent system of system 200 (e.g., an EHR system, a clinical trial design assessment system). In certain cases, data structure 210 is stored in a system external to system 200 and the parent system of system 200. For example, data structure 210 may be stored in a system operated by a laboratory vendor, a healthcare facility, or another third party. In some such cases, module 206 may be configured to communicate with data structure 210 through a communication network (e.g., the Internet).

In some embodiments, system 200 comprises one or more additional modules. In some embodiments, system 200 further comprises a mapping module (not shown in FIG. 2) configured to associate the laboratory result segment with the standard laboratory result identifier to generate a mapped laboratory result segment. In some embodiments, identifier extraction module 206 may associate the laboratory result segment with the standard laboratory result identifier to generate a mapped laboratory result segment. In some embodiments, system 200 further comprises an application module (not shown in FIG. 2) configured to facilitate use of the mapped laboratory result segment during clinical treatment of a patient and/or assessment of one or more aspects of clinical trial design or performance. In certain embodiments, the application module may provide the mapped laboratory result segment to an interface that is accessible to healthcare providers, clinical researchers, and/or patients. In some embodiments, system 200 further comprises an attribute extraction module (not shown in FIG. 2) configured to receive a standard laboratory result identifier (e.g., a LOINC code) from identifier extraction module 206 and identify one or more attributes associated with the standard laboratory result identifier.

Some aspects are directed to at least one non-transitory computer-readable storage medium storing processor-executable instructions that, when executed by at least one hardware processor, cause the at least one hardware processor to perform a method described herein (e.g., method 100).

Some aspects are directed to methods of mapping laboratory results in electronic messages that do not comprise a laboratory result identifier (e.g., a standard laboratory result identifier, a source-specific laboratory result identifier).

In some embodiments, a method comprises receiving a plurality of electronic messages, wherein a first electronic message of the plurality of electronic messages comprises a laboratory result segment comprising one or more field values associated with a laboratory result. In some embodiments, the method further comprises generating a request for a map between the one or more field values and a standard laboratory result identifier. In some embodiments, the method further comprises receiving, in response to the request, the map between the one or more field values and the standard laboratory result identifier. In some embodiments, the method comprises associating one or more additional laboratory result segments that comprise the one or more field values with the standard laboratory result identifier to generate one or more additional mapped laboratory result segments. In some embodiments, one or more steps of the method may be performed using at least one hardware processor.

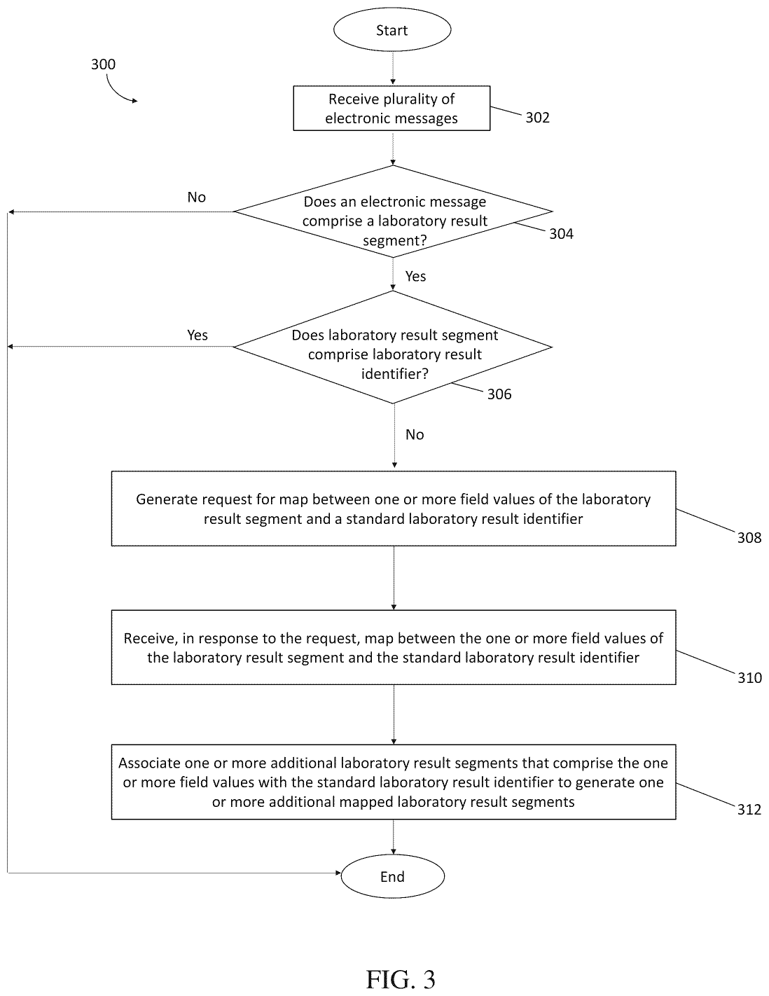

FIG. 3 shows a flow diagram of exemplary method 300 of mapping a plurality of laboratory results in a plurality of electronic messages, according to some embodiments. In some embodiments, some or all steps of method 300 may be performed using at least one hardware processor.

Method 300 begins at block 302, which comprises receiving the plurality of electronic messages. In some embodiments, the plurality of electronic messages are received by a system (e.g., an EHR system, a clinical trial design assessment system) from one or more laboratory test vendors (e.g., Quest Diagnostics, Labcorp, Orchard Laboratories, BioReference Laboratories) and/or one or more healthcare facilities (e.g., hospitals, healthcare clinics, doctors' offices). In some embodiments, the plurality of electronic messages comprises a first electronic message, which may conform to any messaging standard described herein. In certain embodiments, the first electronic message is an HL7 message (e.g., an HL7 v.2x message). The first electronic message may comprise one or more segments, one or more fields, and one or more field values, as described herein.

When the first electronic message is received, method 300 proceeds to block 304, which comprises determining whether the first electronic message comprises a laboratory result segment (e.g., a portion of an electronic message comprising a laboratory result). Determining whether the first electronic message comprises a laboratory result segment may be performed according to any method described herein. In some embodiments, it may be determined that the first electronic message comprises a laboratory result segment. In certain embodiments where the first electronic message is an HL7 segment, the laboratory result segment may comprise an OBX segment (and, in some cases, one or more additional segments, such as associated OBR and/or NTE segments).

When it is determined that the first electronic message comprises a laboratory result segment, method 300 proceeds to block 306, which comprises determining whether the laboratory result segment comprises a laboratory result identifier (e.g., a standard laboratory result identifier, a source-specific laboratory result identifier). Determining whether the laboratory result segment comprises a laboratory result identifier may be performed according to any method described herein.

When it is determined that the laboratory result segment does not comprise a laboratory result identifier, method 300 proceeds to block 308, which comprises generating a request for a map between one or more field values of the laboratory result segment associated with the laboratory result and a standard laboratory result identifier (e.g., a LOINC code). In some cases, a laboratory result segment comprises a plurality of field values associated with the laboratory result. The plurality of field values may comprise one or more attributes of the laboratory result, including but not limited to a laboratory result value, units of the laboratory result value, a reference range, etc. In some cases, the generated request may be for a map between a standard laboratory result identifier and all field values of the laboratory result segment associated with the laboratory result (e.g., the one or more field values of the laboratory result segment may comprise all field values of the laboratory result segment associated with the laboratory result). In certain embodiments where the laboratory result segment comprises an OBX segment, the one or more field values may comprise all field values of the OBX segment. In some cases, the generated request may be for a map between a standard laboratory result identifier and a subset of the plurality of field values of the laboratory result segment associated with the laboratory result (e.g., the one or more field values of the laboratory result segment may comprise a subset of all field values of the laboratory result segment associated with the laboratory result). In certain embodiments where the laboratory result segment comprises an OBX segment, the one or more field values may comprise a subset of field values of the OBX segment.

In method 300, step 310 comprises receiving, in response to the generated request, the map between the one or more field values associated with the laboratory result of the laboratory result segment and the standard laboratory result identifier (e.g., a LOINC code). The map may have been generated by an algorithm and/or by a human (e.g., a physician, a clinical researcher, an informaticist). In certain embodiments, step 310 further comprises associating the laboratory result segment with a system-specific laboratory result identifier (e.g., a laboratory result identifier unique to the system receiving the electronic messages, such as an EHR system or a clinical trial design assessment system).

In method 300, step 312 comprises associating one or more additional laboratory result segments that comprise the one or more field values associated with the laboratory result with the standard laboratory result identifier to generate one or more additional mapped laboratory result segments. In certain embodiments, step 312 further comprises associating the one or more additional laboratory result segments with the system-specific laboratory result identifier.

In some embodiments, method 300 further comprises using the mapped laboratory result segments during clinical treatment of one or more patients and/or assessment of one or more aspects of clinical trial design or performance. The mapped laboratory result segments may be used during clinical treatment of one or more patients and/or assessment of one or more aspects of clinical trial design or performance as described herein.

In some embodiments, method 300 further comprises identifying one or more laboratory result attributes associated with the standard laboratory result identifier (e.g., a LOINC code). The standard laboratory result identifier may be associated with any number and any type of laboratory result attributes described herein.

In some embodiments, one or more steps of method 300 may be omitted.

Some aspects are directed to systems for mapping laboratory results in electronic messages that do not comprise laboratory result identifiers. In some embodiments, a system comprises at least one hardware processor and at least one non-transitory computer-readable storage medium storing processor-executable instructions that, when executed by the at least one hardware processor, cause the at least one hardware processor to perform a method described herein (e.g., method 300). In certain embodiments, the system is (or is a component of) an electronic health record (EHR) system and/or a clinical trial design assessment system. In some embodiments, the system comprises a message ingestion module, a message parsing module, an identifier extraction module, and/or a mapping request module.

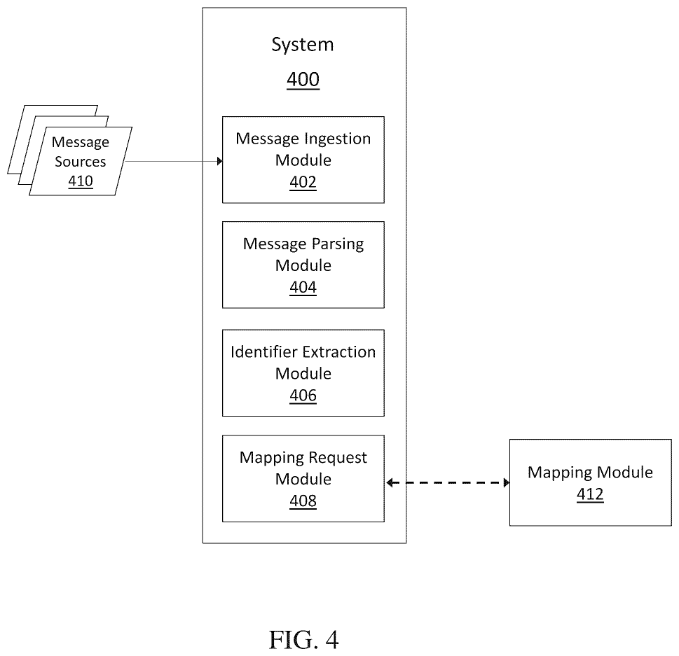

FIG. 4 shows a block diagram of an exemplary system 400 for mapping laboratory results in electronic messages that do not comprise laboratory result identifiers. In some embodiments, system 400 comprises message ingestion module 402, message parsing module 404, identifier extraction module 406, and/or mapping request module 408.

In certain embodiments, message ingestion module 402 is configured to receive electronic messages from message sources 410. Message sources 410 may comprise one or more laboratory vendors and/or one or more healthcare facilities. In some cases, electronic messages may be transmitted from message sources 410 to message ingestion module 402 through a communication network (e.g., the Internet).

In certain embodiments, message parsing module 404 is configured to receive electronic messages from message ingestion module 402 and parse the electronic messages into separate components (e.g., segments, field values). In some embodiments, message parsing module 404 is configured to determine whether an electronic message received from message ingestion module 402 comprises one or more laboratory result segments, as described herein, and to send any laboratory result segments to identifier extraction module 406. In some embodiments, a separate module (not shown in FIG. 4) may be configured to receive the separate components (e.g., segments, field values) of electronic messages from message parsing module 404, determine whether the components comprise one or more laboratory result segments, and send any laboratory result segments to identifier extraction module 406.

In some embodiments, identifier extraction module 406 is configured to receive a laboratory result segment from message parsing module 404 (or, in some cases, another module of system 400) and extract any laboratory result identifiers that may be present in the laboratory result segment according to any method described herein. If no laboratory result identifiers are present in the laboratory result segment, identifier extraction module 406 may send the laboratory result segment to mapping request module 408.

In certain embodiments, mapping request module 408 is configured to receive a laboratory result segment from identifier extraction module 406 and generate a request to map one or more field values of the laboratory result segment associated with a laboratory result to a standard laboratory result identifier. In some instances, the request is sent to optional mapping module 412 to generate the requested mapping. In some instances, the request is sent to one or more users (e.g., a physician, an informaticist, a clinical researcher) to generate the requested mapping.

In some embodiments, system 400 comprises one or more additional modules. In some embodiments, system 400 further comprises a mapping module (not shown in FIG. 4) configured to associate the laboratory result segment with the standard laboratory result identifier to generate a mapped laboratory result segment. In some embodiments, mapping request module 408 may associate the laboratory result segment with the standard laboratory result identifier to generate a mapped laboratory result segment. In some embodiments, system 400 further comprises an application module (not shown in FIG. 4) configured to facilitate use of the mapped laboratory result segment during clinical treatment of a patient and/or assessment of one or more aspects of clinical trial design or performance. In certain embodiments, the application module may provide the mapped laboratory result segment to an interface that is accessible to healthcare providers, clinical researchers, and/or patients. In some embodiments, system 400 further comprises an attribute extraction module (not shown in FIG. 4) configured to receive a standard laboratory result identifier (e.g., a LOINC code) from mapping request module 408 and identify one or more attributes associated with the standard laboratory result identifier.

Some aspects are directed to at least one non-transitory computer-readable storage medium storing processor-executable instructions that, when executed by at least one hardware processor, cause the at least one hardware processor to perform a method described herein (e.g., method 300).

Having thus described several aspects of at least one embodiment of the technology described herein, it is to be appreciated that various alterations, modifications, and improvements will readily occur to those skilled in the art.

Such alterations, modifications, and improvements are intended to be part of this disclosure, and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. As used herein, the term “computer-readable storage medium” encompasses only a non-transitory computer-readable medium that can be considered to be a manufacture (i.e., article of manufacture) or a machine. Alternatively or additionally, aspects of the technology described herein may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but may be distributed in a modular fashion amongst a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which examples are provided herein including with reference to FIGS. 1 and 3. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by an “actor” or a “user”. It should be appreciated that an “actor” or a “user” need not be a single individual, and that in some embodiments, actions attributable to an “actor” or a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A method, comprising:
using at least one hardware processor to perform:
receiving an electronic message comprising a laboratory result segment comprising a laboratory result;
identifying a standard laboratory result identifier associated with the laboratory result segment; and
associating the laboratory result segment with the standard laboratory result identifier to generate a mapped laboratory result segment.

2. The method according to claim 1, wherein the electronic message is an HL7 message.

3. The method according to claim 2, wherein the laboratory result segment comprises an OBX segment.

4. The method according to claim 1, wherein the standard laboratory result identifier is a LOINC code.

5. The method according to claim 1, wherein identifying the standard laboratory result identifier associated with the laboratory result segment comprises searching one or more field values of the laboratory result segment for an occurrence of “LN,” “LOINC,” or “LNC” and identifying the associated LOINC code.

6. The method according to claim 5, wherein searching the one or more field values of the laboratory result segment comprises searching field values of an OBX-3.3 sub-field and/or an OBX-3.6 subfield for an occurrence of “LN,” “LOINC,” and/or “LNC.”

7. The method according to claim 5, wherein identifying the associated LOINC code comprises identifying a field value of an OBX-3.1 and/or OBX-3.4 sub-field as the associated LOINC code.

8. The method according to claim 1, wherein identifying the standard laboratory result identifier comprises identifying a source-specific laboratory result identifier and searching a data structure associating source-specific laboratory result identifiers with standard laboratory result identifiers.

9. The method according to claim 8, wherein identifying the source-specific laboratory result identifier comprises searching one or more field values of the laboratory result segment for an occurrence of “L\hat{ }” or “L|” and identifying the associated source-specific laboratory result identifier.

10. The method according to claim 9, wherein searching the one or more field values of the laboratory result segment comprises searching field values of an OBX-3.3 sub-field and/or an OBX-3.6 subfield for an occurrence of “L\hat{ }” or “L|”.

11. The method according to claim 9, wherein identifying the associated source-specific laboratory result identifier comprises identifying a field value of an OBX-3.1 and/or OBX-3.4 sub-field as the associated source-specific laboratory result identifier.

12. The method according to claim 1, further comprising associating the laboratory result segment with a system-specific laboratory result identifier.

13. The method according to claim 1, further comprising using the mapped laboratory result segment during clinical treatment of a patient and/or assessment of one or more aspects of clinical trial design or performance.

14. The method according to claim 13, wherein using the mapped laboratory result segment during clinical treatment of a patient comprises diagnosing the patient with a disease, suggesting a treatment for the patient, and/or calculating a dosage of a medication based on the mapped laboratory result segment.

15. The method according to claim 13, wherein using the mapped laboratory result segment during assessment of one or more aspects of clinical trial design or performance comprises determining whether a patient falls within a cohort and/or evaluating one or more effects of a medical treatment on a patient enrolled in a clinical trial based on the mapped laboratory result segment.

16. A method, comprising:
using at least one hardware processor to perform:
receiving a plurality of electronic messages, wherein a first electronic message of the plurality of electronic messages comprises a laboratory result segment comprising a laboratory result;
generating a request for a map between one or more field values of the laboratory result segment and a standard laboratory result identifier;
receiving, in response to the request, the map between the one or more field values and the standard laboratory result identifier;
associating the laboratory result segment with the standard laboratory result identifier to generate a first mapped laboratory result segment; and
associating one or more additional laboratory result segments that comprise the one or more field values with the standard laboratory result identifier to generate one or more additional mapped laboratory result segments.

17. The method according to claim 16, wherein the electronic message is an HL7 message.

18. The method according to claim 16, wherein the standard laboratory result identifier is a LOINC code.

19. The method according to claim 16, further comprising associating the first electronic message with a system-specific laboratory result identifier.

20. The method according to claim 19, further comprising associating the one or more additional electronic messages with the system-specific laboratory result identifier.

21. The method according to claim 16, further comprising using the mapped laboratory result segments during clinical treatment of one or more patients and/or assessment of one or more aspects of clinical trial design or performance.

22. The method according to claim 21, wherein using the mapped laboratory result segments during clinical treatment of one or more patients comprises diagnosing one or more patients with a disease, suggesting a treatment for one or more patients, and/or calculating a dosage of a medication based on the mapped laboratory result segments.

23. The method according to claim 21, wherein using the mapped laboratory result segments during assessment of one or more aspects of clinical trial design or performance comprises determining whether a patient falls within a cohort and/or evaluating one or more effects of a medical treatment on a patient enrolled in a clinical trial based on the mapped laboratory result segments.

24. A system, comprising:
at least one hardware processor; and
at least one non-transitory computer-readable storage medium storing processor-executable instructions that, when executed by the at least one hardware processor, cause the at least one hardware processor to perform a method according to claim 1.

25. A system, comprising:
a message ingestion module configured to receive an electronic message;
a message parsing module configured to receive the electronic message from the message ingestion module and parse the electronic message into one or more field values, wherein at least one of the one or more field values comprises a laboratory result; and
an identifier extraction module configured to receive the one or more field values from the message parsing module and identify a standard laboratory result identifier associated with the laboratory result.

26. The system according to claim 25, wherein the identifier extraction module comprises or is in communication with a data structure associating standard laboratory result identifiers and source-specific laboratory result identifiers.

27. A system, comprising:
at least one hardware processor; and
at least one non-transitory computer-readable storage medium storing processor-executable instructions that, when executed by the at least one hardware processor, cause the at least one hardware processor to perform a method according to claim 16.

28. A system, comprising:
a message ingestion module configured to receive an electronic message;
a message parsing module configured to receive the electronic message from the message ingestion module and parse the electronic message into one or more field values, wherein at least one of the one or more field values comprises a laboratory result;
an identifier extraction module configured to receive the one or more field values from the message parsing module and identify any laboratory result identifiers associated with the laboratory result; and
a mapping request module configured to receive the one or more field values and generate a request to map at least one of the one or more field values to a standard laboratory result identifier.

29. At least one non-transitory computer-readable storage medium storing processor-executable instructions that, when executed by at least one hardware processor, cause the at least one hardware processor to perform a method according to claim 1.

30. At least one non-transitory computer-readable storage medium storing processor-executable instructions that, when executed by at least one hardware processor, cause the at least one hardware processor to perform a method according to claim 1.
